(19) United States
(12) Patent Application Publication
(10) Pub. No.: US 2010/0168008 A1
(43) Pub. Date: Jul. 1, 2010

(54) METHOD OF USE OF EPIREGULIN PROTEIN AND NUCLEIC ACID ENCODING THE SAME IN INFLAMMATORY CONDITIONS

(75) Inventors: Muralidhara Padigaru, Mumbai (IN); Somesh Sharma, Mumbai (IN); Arvind Thakkar, Mumbai (IN); Anandharajan Rathina Sabapathy, Mumbai (IN); Sapna Hasit Parikh, Mumbai (IN); Usha Ghosh, Mumbai (IN)

Correspondence Address: MERCHANT & GOULD PC, P.O. BOX 2903, MINNEAPOLIS, MN, 55402-0903, US

(73) Assignee: PIRAMAL LIFE SCIENCES LIMITED., Mumbai (IN)

(21) Appl. No.: 12/515,492

(22) PCT Filed: Nov. 19, 2007
(86) PCT No.: PCT/IB07/54689

§ 371 Date: May 19, 2009

Related U.S. Application Data
(60) Provisional application No. 60/860,139, filed on Nov. 20, 2006.

(51) Int. Cl. A61K 38/18; A61P 29/00; A61P 19/02; A61P 17/06; C12Q 1/68; G01N 33/00

(52) U.S. Cl. 514/12; 435/6; 436/86

(57) ABSTRACT

The present invention relates to nucleotide and protein sequences of epiregulin and epiregulin like proteins and nucleotides and methods employing them as a drug response marker in inflammatory conditions. The invention also provides epiregulin protein for treatment of inflammatory conditions like psoriasis and rheumatoid arthritis alone or in combination with one or more anti-inflammatory compounds.

### FIELD OF INVENTION

The present invention relates to nucleotide and protein sequences of epiregulin and epiregulin like proteins and nucleotides and methods employing them as a drug response marker in inflammatory conditions. The invention also provides epiregulin protein for treatment of inflammatory conditions like psoriasis and rheumatoid arthritis.

### BACKGROUND OF THE INVENTION

Epiregulin is a member of epidermal growth factor (EGF) family of growth factors, which includes, among others, EGF, transforming growth factor (TGF)-alpha, amphiregulin (AR), heparin binding EGF, betacellulin, and the various heregulins. It was first identified in NIH 3T3 cell conditioned medium and is one of seven known ligands for the epidermal growth factor receptor (EGFR) (See Yarden et al., Nat. Rev. Mol. Cell Biol., 2001, 2:127-137). The coding sequence of human epiregulin cDNA predicts a 46-amino acid single-chain polypeptide, exhibiting 24-50% homology with the sequences of other EGFR-ligands.

Epiregulin is synthesized as a transmembrane precursor before being proteolytically cleaved to release a 46-amino-acid active protein. The mode of action of epiregulin is similar to other EGF family members in that it binds to and activates the tyrosine-kinase, ErbB family receptors (ErbB1 through B4). Unlike other EGFR ligands, epiregulin shows dual biological activity; it stimulates proliferation of fibroblasts, hepatocytes, smooth muscle cells, and keratinocytes but inhibits growth of several tumor-derived epithelial cell lines (See Toyoda et al., Biochem. J., 1997, 326:69-75). Epiregulin also shows more potent bioactivity in vitro than other EGF-like growth factors. EGF family members, including epiregulin, play a fundamental role in epithelial tissues and human keratinocytes in an autocrine manner; however, their roles in immune responses and the physiological role of epiregulin remain to be elucidated.

Epiregulin is one of the ligands for the epidermal growth factor receptor (EGFR). The EGF superfamily and EGFR family, including EGFR (ErbB1), ErbB2 (HER2), ErbB3 (HER3), and ErbB4 (HER4) (See Riese & Stern, BioEssays 1998, 20:41-48), play a fundamental role in epithelial tissues. EGF family members, including EGF, transforming growth factor-α, amphiregulin, heparin binding EGF (HB-EGF), epiregulin, and other members, regulate these receptors by inducing their homo- and or hetero-oligomerization (See Yarden & Sliwkowski, Nat. Rev. Mol. Cell Biol., 2001, 2:127-137). EGF family members vary in their ability to activate distinct ErbB heterodimers, and this mechanism may, in part, account for the differences in their bioactivities (See Jackson et al., EMBO J., 2003, 22:2704-2716)

The membrane-anchored precursor of the EGF family is enzymatically processed externally to release a mature soluble form that acts as autocrine and or paracrine growth factor, whereas some members of the EGF family act in the membrane-anchored form. A bioactive transmembrane precursor, pro-HB-EGF, was suggested to induce growth inhibition or apoptosis rather than the proliferative response induced by soluble HB-EGF. Epiregulin acts as an autocrine growth factor in normal human keratinocytes in vitro, in the differentiation of vascular smooth muscle cells and as one of the paracrine mediators that propagate luteinizing hormone signals in the ovarian follicle; however, its physiological role in vivo still remains unknown.

Both the membrane-bound and secreted forms of epiregulin are identified. Sirasawa et al (See PNAS, 2004, 101:13921) have reported that epiregulin deficiency causes chronic dermatitis in mice.

The inflammatory response represents the body's response to injury which can arise from a variety of agents such as infectious organisms, toxins including alcohol, allergies, autoimmune responses, trauma, lacerations, burns, circulatory occlusions and other factors that induce tissue damage. At present, C-reactive protein is used extensively as marker of inflammatory status. However, it is a non-specific marker that cannot diagnose a particular disease but is used to gain insight into conditions of inflammation. More sensitive markers are needed either as individual markers or used in combination with C-reactive protein for greater prognostic value (Philips et al., J. of Endocrinology, 1999; 161, 195-198).

Wound healing is the process of repair that follows injury to the skin and other soft tissues. The process of wound healing involves two essential components—regeneration and repair. In regeneration, a specialized tissue is replaced by the proliferation of surrounding undamaged specialized cells. In repair, lost tissue is replaced by granulation tissue to form scar tissue.

Psoriasis (PS) is a common chronic skin disease characterized by skin inflammation and hyperproliferation of epidermal keratinocytes that can be triggered by both genetic and environmental risk factors. It affects approximately 2% of individuals from the northern European population. The three cardinal features of psoriatic lesions are erythema, indurations and scaling. An association with psoriatic arthritis is seen in 6-10% of cases. In young patients it is frequently triggered by infection with group A streptococci, and can also occur with human immunodeficiency virus (HIV) infection (See Duvic et al., J. Invest. Dermatol., 1990, 95:38 S-40S). The pathogenesis of psoriasis remains to be elucidated.

Rheumatoid arthritis (RA) is a systemic disease with highly variable clinical manifestations. Rheumatoid arthritis generally affects the joints of the extremities and involves synovial joints between two bones. It is a chronic inflammatory disease that goes through several stages and finally results in a massive destruction of joints or inflammations in the tendon area. According to the recent state of knowledge, it is considered that T cells initiate and maintain the inflammation. In this process, cytokines as well as mesenchymal cells (macrophages and synovial fibroblasts) are active. It is very probable that the cytokine TNF-α is one of the mediators of the inflammation. It promotes the formation of the pannus, which is typical of RA, and promotes cartilage destruction. TNF-α increases the number of adhesion molecules for leukocytes on the surface of the endothelial cells and the penetration of the capillary endothelial layer. This results in an increased inflow of leukocytes into the synovia. The cytokine promotes the secretion of matrix metalloproteinases by the synoviocytes. These enzymes are directly involved in the destruction of bones and cartilage. TNF-α also sensitizes pain receptors, which is associated with the induction of pain sensations. TNF-α plays a critical role in the initiation and maintenance of RA.

Changes in gene and/or protein expression have been described previously in psoriasis and other inflammatory diseases. IL-1 and TNF-α are up-regulated in psoriasis and activate several cellular signaling pathways including the NF-κB pathway. Differential gene expression of many transcripts has been reported in psoriatic skin as compared to the normal skin (See Bowcock et al, Human Molecular Genetics, 2001, 10:1793). The modification could be either up-regulation (e.g.: IL-8R) or down-regulation (e.g.: cysteine-rich protein 1).

### SUMMARY OF THE INVENTION

The present invention relates to nucleotide and protein sequences of epiregulin and its use as a drug response marker in inflammatory conditions. Moreover the invention relates to the diagnosis and treatment of inflammatory conditions. The inflammatory conditions of the instant invention may include, but are not limited to rheumatoid arthritis, psoriasis, atopic dermatitis, psoriatic arthritis and juvenile rheumatoid arthritis.

In one aspect the invention provides a method of monitoring drug response in patients receiving treatment for inflammatory condition by checking epiregulin gene expression in the patient's blood sample. In another aspect, the invention provides a method of monitoring drug response in patients receiving treatment for inflammatory condition by checking epiregulin gene expression in the patient's serum sample.

The invention also provides a method of monitoring drug response in patients undergoing treatment for inflammatory condition with anti-inflammatory compounds, the method comprising measuring epiregulin gene expression in the patient's blood or serum sample for elevated levels of the epiregulin gene expression.

Another aspect of the invention is to provide a method of treating a subject suffering from inflammatory conditions comprising administering a therapeutically effective amount of epiregulin protein alone or in combination with one or more anti-inflammatory compounds.

### DETAILED DESCRIPTION OF THE INVENTION

The present invention includes a method of detecting an increase in epiregulin gene expression following administration of anti-inflammatory agent. In one embodiment of the present invention, epiregulin is used as drug response marker for inflammatory conditions. In a further embodiment, epiregulin protein levels are used to diagnose the inflammatory condition in a subject. The invention described herein shows that epiregulin mitogen like growth factor reacts with EGFR and is involved in epithelial cell proliferation and other biological processes. Furthermore, the inventors' studies show that secreted epiregulin is selectively up regulated in response to anti-inflammatory agent, which clearly indicates its use as a drug response marker. In addition, epiregulin regulates wound healing by down-regulation of inflammatory processes and epithelial cell proliferation in keratinocytes, which is indicative of its therapeutic potential for inflammatory conditions like psoriasis.

The length of epiregulin polynucleotide is 506 bp, the accession no. is NM_{—}001432, and the length of protein sequence is 169 amino acids. Epiregulin gene of the present invention has a DNA sequence, encoded by polynucleotide as shown in Table 1 and protein sequence as in Table 2.

TABLE 1
Epiregulin Nucleotide (SEQ ID NO: 1)

agccccagcgcccgccccacgccgATGACCGCGGGGAGGAGGATGGAGAT
GCTCTGTGCCGGCAGGGTCCCTGCGCTGCTGCTCTGCCTGGGTTTCCATC
TTCTACAGGCAGTCCTCAGTACAACTGTGATTCCATCATGTATCCCAGGA
GAGTCCAGTGATAACTGCACAGCTTTAGTTCAGACAGAAGACAATCCACG
TGTGGCTCAAGTGTCAATAACAAAGTGTAGCTCTCATGAATGGCTATTGT
TTGCATGGACAGTGCATCTATCTGGTGGACATGAGTCAAAACACTGCAGG
TGTGAAGTGGGTTATACTGGTGTCCGATGTGAACACTTCTTTTTAACCGC
CACCAACCTTTAAGCAAAGAGTATGTGGCTTTGACCGTGATTCTTATTAT
TTTGTTTCTTATCACAGTCGTCGGTTCCACATATTATTTCTGCAGATGGT
ACAGAAATCGAAAAAGTAAAGAACCAAAGAAGGAATATGAGAGAGTTACC
TCAGGGGATCCAGAGTTGCCGCAAGTCTGAatggcgccatcaaacttatg
ggca

TABLE 2
Epiregulin Protein (SEQ ID NO: 2)

Human Epiregulin protein sequence (NP_001423,
HUMAN_EREG) 169 amino acids
MTAGRRMEMLCAGRVPALLLCLGFHLLQAVLSTTVIPSCIPGESSDNCTA
LVQTEDNPRVAQVSITKCSSDMNGYCLHGQCIYLVDMSQNYCRCEVGYTG
VRCEHFFLTVHQPLSKEYVALTVILIILFLITVVGSTYYFCRWYRNRKSK
EPKKEYERVTSGDPELPQV

The invention also includes epiregulin like genes, their polypeptides, derivatives and fragments that have biological activity similar to that of epiregulin.

The inventors' studies employed a method of first isolating RNA from normal human peripheral blood leukocytes treated with anti-inflammatory drugs (such as those disclosed in published PCT patent application WO2006051477, incorporated herein by reference) and Rolipram. (Rolipram is used as a positive control drug to identify potential therapeutic agents for the treatment of chronic inflammatory disorders such as rheumatoid arthritis.) The isolate leukocytes were activated with bacterial lipopolysaccharide. The quality and quantity of RNA was measured by spectrometry and gel electrophoresis. Gene expression profiling was performed by microarray procedure that is known in the art.

Increased expression of epiregulin in response to anti-inflammatory compound indicates that the polynucleotides and polypeptides of the present invention are useful as drug response markers. The marker can be employed for differential identification of responders versus non-responders to treatment of, for example, inflammation and inflammatory disorders. Epiregulin, which is a secreted protein, can be detected in the blood sample of the subject in measurable quantities. Elevated epiregulin levels in the sample are indicative of positive response to anti-inflammatory compounds.

The polynucleotides and polypeptides of the invention are also useful as diagnostic markers for identification of the disease condition in a given biological sample in conditions that include, but are not limited to, inflammation and inflammatory disorders. Epiregulin can be detected in the blood or serum sample of the subject in measurable quantities. Modified epiregulin levels in the subject's sample are indicative of susceptibility to inflammation or inflammatory conditions.

The present invention includes a method of treating an inflammatory disorder (such as psoriasis) by administering to a subject in need thereof an effective amount of epiregulin protein. The invention also provides administering epiregulin protein in combination with known or anti-inflammatory compounds to alleviate an inflammatory condition. Such combinations include but not limited to known anti-inflammatory drugs such as cyclophosphamide and azathioprine, dexamethasone and hydrocortisone. The two compounds, epiregulin and the anti-inflammatory agent, can be administered once or twice daily as required. The compounds can be administered simultaneously or sequentially. The invention also provides formulating epiregulin with an anti-inflammatory compound and administering to the patient that needs treatment for inflammatory conditions. Although not limiting to the present invention, it is believed that administering epiregulin as a treatment for psoriasis may be based on epiregulin induced epithelial cell proliferation and down regulation of inflammatory processes in the skin. These two features promote wound healing.

By use of the methods of the invention, one skilled in the genomics art can identify multiple groups of related genes, many representing processes with previously unrecognized relationships to immune related or inflammatory conditions. Thus, the invention also provides compositions of matter comprising sets of genes, polynucleotides, polypeptides encoded by said polynucleotides identified as being involved in treating skin diseases, cancer, and inflammatory conditions. In particular, the set of genes can be used for the treatment of inflammatory conditions such as psoriasis and arthritis.

### DEFINITIONS

Biomarker is a well-known and useful concept in the genomic and biopharmaceutical art. “Biomarker” referred to herein, is a measurable biological manifestation that is a quantitative indication of the presence and degree of an underlying biological process of interest e.g.: drug response or diagnosis.

As used herein, a “polynucleotide” refers to a molecule having a nucleic acid sequence contained in SEQ ID NO: 1. For example, the polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence.

Moreover, as used herein, a “polypeptide” refers to a molecule having the translated amino acid sequence generated from the polynucleotide as broadly defined. The polypeptide used in the present invention is exemplified by SEQ ID NO: 2.

In the present invention, a “secreted” protein refers to those proteins capable of being directed to the endoplasmic reticulum, secretory vesicles, or the extracellular space as a result of a signal sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

A polypeptide having “biological” activity refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention, for example, the candidate polypeptide may exhibit greater activity or not more than about 25-fold less and, preferably, not more than about tenfold less activity, and most preferably, not more than about three-fold less activity relative to the polypeptide of the present invention.

The invention is further illustrated in the following non-limiting examples

### EXAMPLES

Compound P979 was synthesized according to processes described in published PCT patent application WO2006051477, incorporated herein by reference. P979 is an anti-inflammatory compound demonstrated to have an IC₅₀ less than 1 μM as reported in Example 43, Table 2 of the PCT application WO2006051477.

### Example 1

### Epiregulin Expression in Peripheral Blood Monocytes (PBMNCs) After Exposure to Anti-Inflammatory Agent and its Study by Microarray Based Expression Profiling

### Method:

Exposure of PBMCs to P979 and Rolipram: Human peripheral blood leukocytes were obtained from normal blood donors by density gradient separation, washed with PBS (phosphate buffered saline), and plated on petri dishes using RPMI medium. Cells were treated for 30 minutes with test compounds at concentrations 0.3, 1.0 and 3.0 μM and with Rolipram at 300 μM as a control as shown in the results section and subsequently activated with bacterial lipopolysaccharide (LPS). After 2 hours, cells were harvested and RNA was prepared.

Preparation of RNA: RNA was prepared by adding 1 ml of trizol reagent (Sigma) subsequently purified using Qiagen-RNAEasy kit, using the instruction from manufacturer's protocol. Quality and quantity of RNA was measured by spectrometry and gel electrophoresis.

Gene expression profiling by microarray: A total of 10 μg of RNA was used to generate cRNA, which is subsequently labeled with Cy3 or Cy5 fluorescent dyes. Control and test cRNAs labeled with Cy3 and Cy5 respectively were pooled and hybridized to DNA chip. DNA chips were synthesized in house and contains 23, 290 oligomers (70-mer oligonucleotides, obtained from Illumina, U.S.A.) that represent well-annotated reference gene list from human genome. Pronto plus microarray kit from Promega was used throughout the microarray procedure according to the manufacturer's protocol. Hybridized DNA chips were scanned and images were generated. Following image analysis, image intensity for each of the 23, 290 spots were measured for test and control slides.

### Results:

Gene expression was expressed as a ratio of the image intensity between test and control slide for a given gene (Table 3). Ratio of intensity of one between normal and test slide was considered as normal, whereas ratio of 0.5 and less was considered as down-regulated expression. A ratio of 1.5 and above was considered as gene up-regulation.

TABLE 3
EREG expression in PBMCs after exposure to anti-inflammatory
agent as studied by microarray based expression profiling

| | P979 | | | Rolipram |
|---|---|---|---|---|
| Drug concentration | 0.3 μM | 1.0 μM | 3.0 μM | 300 μM |
| Ratio intensity | 1.42 | 2.63 | 2.95 | 1.03 |

As shown in the above data, the expression of epiregulin gene is up-regulated selectively by anti-inflammatory agent in a concentration dependant manner, but not by the control compound Rolipram.
Confirmation of Data Obtained from Microarray:

Real Time Polymerase Chain Reaction (RT-PCR): To confirm the data obtained from microarray, we performed RT-PCR using specific primers for epiregulin gene.

EREG expression in PBMCs after exposure to anti-inflammatory agent was confirmed by real time polymerase chain reaction (RT-PCR). Microarray based gene expression profiling studied large-scale gene expression. RT PCR focused on the gene/genes of interest and expression analysis was carried out under similar experimental conditions.

| | |
|---|---|
| Left primer | CGTGTGGCTCAAGTGTCAAT; |
| Right primer | TGGAACCGACGACTGTGATA; |

Product size: 235 base pairs.

TABLE 4

DNA sequences for primers are represented in bold, italics and
underline in the epiregulin DNA sequence. Human Epiregulin DNA
sequence (NM_001432, HUMAN_EREG)

| | |
|---|---|
| 1 | agccccagcgcccgctcccatcgccgATGACCGCGGGGAGGAGGATGGAGATGCTCTGTG |
| 61 | CCGGCAGGGTCCCTGCGCTGCTGCTCTGCCTGGGTTTCCATCTTCTACAGGCAGTCCTCA |
| 121 | GTACAACTGTGATTCCATCATGTATCCCAGGAGAGTCCAGTGATAACTGCACAGCTTTAG |
| 181 | TTCAGACAGAAGACAATCCA [symbol] AACAAAGTGTAGCTCTGACA |
| 241 | TGAATGGCTATTGTTTGCATGGACAGTGCATCTATCTGGTGGACATGAGTCAAAACTACT |
| 301 | GCAGGTGTGAAGTGGGTTATACTGGTGTCCGATGTGAACACTTCTTTTTAACCGTCCACC |
| 361 | AACCTTTAAGCAAAGAGTATGTGGCTTTGACCGTGATTCTTATTATTTTGTTTCT [symbol] |
| 421 | [symbol] CATATTATTTCTGCAGATGGTACAGAAATCGAAAAAGTAAAGAAC |
| 481 | CAAAGAAGGAATATGAGAGAGTTACCTCAGGGGATCCAGAGTTGCCGCAAGTCTGAatgg |
| 541 | cgccatcaaacttatgggca |

The aliquot of RNA samples as used for microarray study was used for RT-PCR analysis. The experiment was carried out using cMaster™ RTplusPCR System kit from Eppendorf using the protocol as provided in the kit. Briefly, cDNA was synthesized using RNA from various sources as shown in the results and subsequently quantified during amplification step by incorporation of Syber green dye into the amplicons.

The RT-PCR analysis confirmed the finding of specific activation of EREG in response to P979 at certain drug concentrations (Table 5)

TABLE 5
EREG Expression in PBMC following treatment
with P979 and Rolipram

| | Concentration | Log-fold |
|---|---|---|
| Drug Used | (μM) | Change |
| P979 | 0.3 | 0.93 |
| | 0.3 | 1.3 |
| | 1 | 3.4 |
| | 3 | 3.2 |
| | 3 | 2.0 |
| Rolipram | 10 | 1.2 |

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

We claim:

1. A method for predicting the response of a patient to a drug employed in the treatment of inflammatory condition in the patient, the method comprising: determining the level of epiregulin gene expression in the patient's blood sample.

2. The method of claim 1, wherein the level of the epiregulin gene expression is elevated in the patient's blood sample.

3. The method of claim 1, wherein the inflammatory condition comprises rheumatoid arthritis, psoriasis, atopic dermatitis, psoriatic arthritis, juvenile rheumatoid arthritis, or combination thereof.

4. A method for monitoring drug response in patients receiving treatment for inflammatory condition, the method comprising: determining level of epiregulin protein in the patient's serum sample.

5. The method of claim 4, wherein the level of the epiregulin protein is elevated in the patient's serum sample.

6. The method of claim 4, wherein the inflammatory condition comprises rheumatoid arthritis, psoriasis, atopic dermatitis, psoriatic arthritis, juvenile rheumatoid arthritis, or combination thereof.

7. A method of determining the likelihood of a subject developing inflammatory condition, the method comprising: determining level of epiregulin gene expression in the subject's blood sample.

8. A method of treating a subject suffering from inflammatory condition comprising: administering therapeutically effective amount of epiregulin protein alone or in combination with at least one anti-inflammatory compound.

9. The method of claim 8, wherein the epiregulin is administered as oral, parenteral, subcutaneous or topical formulation.

10. The method of claim 8, wherein the inflammatory condition comprises rheumatoid arthritis, psoriasis, atopic dermatitis, psoriatic arthritis, juvenile rheumatoid arthritis, or combination thereof.

11. A method of monitoring drug response in a patient receiving treatment for inflammatory condition with anti-inflammatory compounds, the method comprising: measuring epiregulin gene expression in the patient's blood or serum sample for elevated levels of the epiregulin gene expression.


SEQUENCE LISTING (5 sequences)

SEQ ID NO: 1 - DNA, 556 bp, Homo sapiens
  misc_feature: Epiregulin Nucleotide as coded by Seq ID No. 1
agccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctcatgaatggctattgtttgcatggacagtgcatctatctggtggacatgagtcaaaacactgcaggtgtgaagtgggttatactggtgtccgatgtgaacacttctttttaaccgccaccaacctttaagcaaagagtatgtggctttgaccgtgattcttattattttgtttcttatcacagtcgtcggttccacatattatttctgcagatggtacagaaatcgaaaaagtaaagaaccaaagaaggaatatgagagagttacctcaggggatccagagttgccgcaagtctgaatggcgccatcaaacttatgggca

SEQ ID NO: 2 - protein, 169 aa, Homo sapiens
  MISC_FEATURE: Epiregulin Protein coded as Seq ID No. 2
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 3 - DNA, 20 bp, Homo sapiens
  misc_feature: Left primer
cgtgtggctcaagtgtcaat

SEQ ID NO: 4 - DNA, 20 bp, Homo sapiens
  misc_feature: Right primer
tggaaccgacgactgtgata

SEQ ID NO: 5 - DNA, 560 bp, Homo sapiens
  misc_feature: Human Epiregulin DNA sequence
agccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtgtgaagtgggttatactggtgtccgatgtgaacacttctttttaaccgtccaccaacctttaagcaaagagtatgtggctttgaccgtgattcttattattttgtttcttatcacagtcgtcggttccacatattatttctgcagatggtacagaaatcgaaaaagtaaagaaccaaagaaggaatatgagagagttacctcaggggatccagagttgccgcaagtctgaatggcgccatcaaacttatgggca